(19) United States
(12) Patent Application Publication
(10) Pub. No.: US 2022/0273739 A1
(43) Pub. Date: Sep. 1, 2022

(54) COMBINATION DRUG FOR TREATING MALIGNANT TUMOR, PHARMACEUTICAL COMPOSITION FOR TREATING MALIGNANT TUMOR, AND PHARMACEUTICAL COMPOSITION FOR MALIGNANT TUMOR TREATMENT

(71) Applicant: National University Corporation Tokai National Higher Education and Research System, Aichi (JP)

(72) Inventors: Hideki KASUYA, Nagoya-shi, Aichi (JP); Yoshinori NAOE, Nagoya-shi, Aichi (JP); Shigeru MATSUMURA, Nagoya-shi, Aichi (JP); Ibrahim Ragab Nassr EISSA, Nagoya-shi, Aichi (JP)

(21) Appl. No.: 17/631,808

(22) PCT Filed: Jul. 30, 2020
(86) PCT No.: PCT/JP2020/029197

§ 371 Date: Jan. 31, 2022

(30) Foreign Application Priority Data
Aug. 5, 2019 (JP) 2019-143949

(51) Int. Cl. A61K 35/763; A61K 31/7084; A61P 35/00

(52) U.S. Cl. CPC A61K 35/763; A61K 31/7084; A61P 35/00

(57) ABSTRACT

Provided are a combination drug for treating a malignant tumor, a pharmaceutical composition for treating a malignant tumor, and a pharmaceutical composition for malignant tumor treatment that have an exceptional antitumor effect. A malignant tumor can be treated by a combination drug containing a first medicine that includes an oncolytic virus belonging to the herpes simplex viruses type 1 as an active ingredient, and a second medicine that includes an interferon gene stimulating factor agonist as an active ingredient.

### TECHNICAL FIELD

The disclosure of the present application relates to a concomitant drug for treating a malignant tumor, a pharmaceutical composition for treating a malignant tumor, and a malignant tumor-treating pharmaceutical composition.

### BACKGROUND ART

Although malignant tumors (cancer) (hereafter, also simply referred to as “tumor”) are the leading cause of Japanese mortality, it is said that the mortality can be significantly reduced if early treatment with early diagnosis is possible. As treatment of tumors, treatment by surgical resection, radiation, an anti-cancer drug, and the like are known. Further, in recent years, tumor treatment using an oncolytic virus that specifically multiplies in tumor cells is also known.

As the oncolytic virus, several viruses derived from adenovirus or other virus species are known, and an example thereof may be, for example, Canerpaturev (hereafter, also referred to as “C-REV”). The C-REV is an oncolytic herpes simplex virus (HSV), which is a mutant virus that infects only tumors and thereby destroys the tumors to develop an antitumor effect. Furthermore, such viruses are spread from the tumor destroyed by virus infection, and released cancer antigens cause activation of tumor-specific lymphocytes. Such activated lymphocyte has strong tumor immunity. Thus, it is known that, when the C-REV is administered to a tumor, the antitumor effect caused by tumor-specific lymphocytes instead of by virus infection is developed against not only tumor cells actually infected by C-REV but also tumor cells around the tumor cells destroyed by the C-REV and even distantly metastasized tumor cells (see Non-Patent Literature 1).

Further, it is known that activation of cyclic GMP-AMP synthase (cGAS) that is a double strand DNA sensor in an innate immune system and further activation of stimulator of interferon genes (hereafter, also referred to as “STING”) cause Type-I interferon (Type-I IFN) to be induced. The cGAS is recognized as a member of monitoring mechanisms in host cell that sense infection by binding to an intracellular pathogen and a virus-derived short DNA and, in response thereto, produce cyclic GMP-AMP (hereafter, also referred to as “cGAMP”), and induce production of Type-I IFN via activation of the STING. The produced Type-I IFN serves as an alert to surrounding cells and, at the same time, causes its own infection defense gene response. Further, the cGAS act on immune cells to activate defensive pathogen specific immune response of a pathogen specific antibody and an antigen specific helper T cell and a cytotoxic T cell. Further, it is also known that STING activation of an antigen-presenting cell increases the antigen-presenting ability and activates tumor immunity, and this leads to an antitumor effect (see Non-Patent Literature 2).

Furthermore, with respect to treatment of tumor including administration of an oncolytic virus, one proposed method is to determine function activity of STING or cGAS in a cell isolated from human subject having a tumor and select the oncolytic virus as a treatment method when the function activity is defective (see Patent Literature 1).

### CITATION LIST

### Non-Patent Literature

Non-Patent Literature 1: Yoshihiro Hotta et al., “Curative effect of HF10 on liver and peritoneal metastasis mediated by host antitumor immunity”, Oncolytic Virotherapy, 2017:6, 31-38
Non-Patent Literature 2: Tiejun Liet et al., “Antitumor Activity of cGAMP via Stimulation of cGAS-cGAMPSTING-IRF3 Mediated Innate Immune Response”, Scientific Reports, 6:19049, D01:10.1038/srep19049
Non-Patent Literature 3: Yoko Ushijima et al., “Determination and analysis of the DNA sequence of highly attenuated herpes simplex virus type 1 mutant HF10, a potential oncolytic virus”, Microbes and Infection, 9 (2007), 142-149

### PATENT LITERATURE

Patent Literature 1: Japanese Patent Application Laid-Open No. 2018-519278

### SUMMARY OF INVENTION

### Technical Problem

It is known that a C-REV administered to a tumor develops an antitumor effect as disclosed in Non-Patent Literature 1 and that activation of a STING induces an antitumor effect as disclosed in Non-Patent Literature 2. However, the C-REV disclosed in Non-Patent Literature 1 does not infect normal cells, and viruses and cancer antigens released from a tumor destroyed by the C-REV develop an antitumor effect against surrounding tumor cells. Thus, an environment in which viruses easily multiply inside a tumor cell is more preferable than an environment in which multiplication of tumor cells is suppressed, because viruses and cancer antigens are continuously released. On the other hand, the STING activation disclosed in Non-Patent Literature 2 is to administer a STING agonist to a tumor to activate an immune response and exhibits an activation effect of antivirus immunity and antitumor immunity. That is, this creates an environment in which multiplication of tumor cells is suppressed and, at the same time, viruses are eradicated. According to Patent Literature 1, however, it is considered that a tumor with a reduced function of a STING pathway has high sensitivity to an oncolytic virus and a higher treatment effect is expected from administration of the virus.

As described above, while various methods for treatment of tumors have been reported, more intensive and reliable treatment of tumors is desired. According to a thorough study regarding more reliable treatment of tumors, the disclosure of the present application has newly found that (1) combined use of two drugs of an oncolytic virus and a STING agonist that have completely different antitumor action mechanisms against a tumor cell surprisingly develops synergy thereof despite the fact that the actions of the two drugs appear to be antagonistic and the effects thereof are expected to be cancelled and furthermore that (2), in addition to improvement of the antitumor effect on a tumor at a site where a drug was administered, an antitumor effect on a tumor at a site distant from the tumor at the site where the drug was administered is synergistically improved.

That is, an object of the disclosure of the present application is to provide a concomitant drug for treating a malignant tumor, a pharmaceutical composition for treating a malignant tumor, and a malignant tumor-treating pharmaceutical composition that are superior in an antitumor effect.

### Solution to Problem

The disclosure of the present application relates to a concomitant drug for a treating malignant tumor, a pharmaceutical composition for a treating malignant tumor, and a malignant tumor-treating pharmaceutical composition illustrated below.

(1) A concomitant drug for treating a malignant tumor, the concomitant drug comprising:

a first medicine containing an oncolytic virus belonging to a herpes simplex virus type 1 as an active component; and

a second medicine containing a stimulator of interferon genes agonist as an active component.

(2) The concomitant drug according to (1) above, wherein the oncolytic virus is a mutant virus not artificially modified.
(3) The concomitant drug according to (1) or (2) above, wherein the stimulator of interferon genes agonist is 2′3′-cyclic GMP-AMP.
(4) The concomitant drug according to any one of (1) to (3) above, wherein the first medicine and the second medicine are administered to a malignant tumor.
(5) A pharmaceutical composition for treating a malignant tumor, the pharmaceutical composition comprising, as active components, a combination of:

an oncolytic virus belonging to a herpes simplex virus type 1; and

a stimulator of interferon genes agonist,

wherein the pharmaceutical composition is used to be administered to a patient in order of the oncolytic virus belonging to the herpes simplex virus type 1 and the stimulator of interferon genes agonist.

(6) The pharmaceutical composition according to (5) above, wherein the oncolytic virus is a mutant virus not artificially modified.
(7) The pharmaceutical composition according to (5) or (6), wherein the stimulator of interferon genes agonist is 2′3′-cyclic GMP-AMP.
(8) The pharmaceutical composition according to any one of (5) to (7) above, wherein the oncolytic virus and the stimulator of interferon genes agonist are administered to a tumor.
(9) A malignant tumor-treating pharmaceutical composition containing, as an active component, an oncolytic virus belonging to a herpes simplex virus type 1 for use in combined therapy with a malignant tumor-treating pharmaceutical composition containing a stimulator of interferon genes agonist as an active component.
(10) A malignant tumor-treating pharmaceutical composition containing, as an active component, a stimulator of interferon genes agonist for use in combined therapy with a malignant tumor-treating pharmaceutical composition containing an oncolytic virus belonging to a herpes simplex virus type 1 as an active component.

### Advantageous Effect

Treatment of a tumor by using the concomitant drug for treating a malignant tumor, the pharmaceutical composition for treating a malignant tumor, and the malignant tumor-treating pharmaceutical composition disclosed in the present application improves an antitumor effect on a tumor at a site where the drug was administered and also improves an antitumor effect on a tumor at a site distant from the tumor at the site where the drug was administered.

### BRIEF DESCRIPTION OF DRAWINGS

FIG. 1A is a graph illustrating the antitumor effect in a treated group with administration of a drug in Example 1 and Comparative examples 1 to 3.

FIG. 1B is a graph illustrating the antitumor effect in a tumor without administration of a drug (non-treated group), which is a tumor spaced apart from the treated group with administration of the drug, in Example 1 and Comparative examples 1 to 3.

FIG. 2 is a graph illustrating the antitumor effect in a tumor (tumor without administration of the drug) spaced apart from the tumor with administration of the drug in Example 2 and Comparative examples 4 to 5.

### DESCRIPTION OF EMBODIMENTS

A concomitant drug for treating a tumor (hereafter, also simply referred to as “concomitant drug”), a pharmaceutical composition for treating a tumor (hereafter, also simply referred to as “pharmaceutical composition”), and a tumor-treating pharmaceutical composition disclosed in the present application will be described below in detail.

### [Embodiment of Concomitant Drug]

First, the embodiment of the concomitant drug will be described. The embodiment of the concomitant drug includes a first medicine containing an oncolytic virus belonging to a herpes simplex virus type 1 (HSV-1) as an active component and a second medicine containing a stimulator of interferon genes (STING) agonist as an active component.

The oncolytic virus belonging to the herpes simplex virus type 1 contained in the first medicine is not particularly limited as long as it has a property of specifically multiplying inside a tumor and may be a naturally isolated virus strain or may be an artificially modified virus strain (to which a heterologous gene has been introduced, for example). The oncolytic virus belonging to the herpes simplex virus type 1 may be, for example, Canerpaturev (C-REV, former name: HF10). The C-REV is an attenuated mutant strain of the herpes simplex virus type 1 that has not been artificially modified and holds a functional _{γ1}134.5 gene, and an antitumor action is obtained by administration of the C-REV to a local tumor. The C-REV is given by Takara Bio Inc. for the purpose of joint research. The C-REV in the present specification is a herpes simplex virus reported by International Publication No. WO2002/092826, Hepato-Gastroenterology 2003; 50: 961-966, Microbes Infect. 2007; 1-8, Current Gene Therapy. 2008 June; 8(3): 208-21, Front Oncol. 2017; 7, 149, or the like.

The oncolytic virus belonging to the herpes simplex virus type 1 other than the C-REV may be, for example, talimogene laherparepvec (T-VEC: Immunotherapy. 2015, 7, (6): 611-9). The T-VEC is an oncolytic virus derived from a herpes simplex virus type 1 mounted with a GM-CSF gene and is approved as a medicine by the U.S. Food and Drug Administration. Further, G47Δ is also an oncolytic virus created by modifying a gene of a herpes simplex virus type 1, and the configuration thereof has also been reported (Proc Natl Acad Sci USA. 2001, 98 (11): 6396-401). JS1/34.5-/47-/mGM-CSF is a herpes simplex virus type 1, which lacks ICP34.5 and ICP47 and has an inserted gene that codes mGM-CSF, and is reported to have a strong oncolytic property (Gene Therapy 2003, 10(4); 292-303).

Furthermore, hrR3 is also known as an oncolytic virus created by modifying a gene of the herpes simplex virus type 1. The hrR3 is a recombinant HSV having inserted lacZ gene in the coding sequence of the ICP6. The creation method of the hrR3 is disclosed in DAVID J. GOLDSTEIN et al., “Herpes Simplex Virus Type 1-Induced Ribonucleotide Reductase Activity Is Dispensable for Virus Growth and DNA Synthesis: Isolation and Characterization of an ICP6 lacZ Insertion Mutant”, JOURNAL OF VIROLOGY, January 1988, p. 196-205. The hrR3 used in Examples described later was created by the procedure disclosed in the above paper.

The dosage form of the first medicine is not particularly limited as long as the effect of the contained oncolytic virus is developed. In a case of an oncolytic virus administered locally to a tumor, since administration through injection is the simplest way, injections are preferable as a dosage form. A liquid medium used for forming an injection is not particularly limited as long as it does not affect an organism, and a known pharmaceutical liquid medium, for example, a saline solution or the like may be used. Further, the first medicine may contain a conventionally used additive agent, if necessary. The additive agent may be, for example, an existing additive agent such as an excipient, a binder, a lubricant, a disintegrator, a corrective, a solvent, a stabilizer, a base, a humectant, a preservative, or the like but is not limited thereto. Furthermore, the first medicine may also be a solid formulation that can take a solution form when dosed (for example, a freeze-dried formulation).

The STING agonist contained in the second medicine is not particularly limited as long as it can activate the STING. For example, various cyclic dinucleotides (cyclic-di-AMP, cyclic-di-GMP, or the like) may be used, and 2′3′-cyclic GMP-AMP (hereafter, also referred to as “cGAMP”) is preferable. Once intracellular DNA is recognized by a cyclic GMP-AMP synthase (cGAS), the cGAMP is generated as a second messenger. The cGAMP is available from catalog No, tlrl-nacga 23 by Invivogen. Further, many cyclic dinucleotides having STING activation action including ADU-S100, which is a cyclic dinucleotide formulation by Novaltis, and the manufacturing method thereof have been reported (for example, International Publication Nos. WO2015/185565, WO2017/123669, WO2018/208667, and the like). Such artificially synthesized cyclic dinucleotides include cyclic dinucleotides to which phosphorothioate bond has been introduced. ADU-S100 that is one of those cyclic dinucleotides is available as catalog No. CT-ADUS100 by ChemieTek. Furthermore, a derivative of the above cyclic dinucleotides (for example, prodrug) may be used as an active component of the second medicine.

The dosage form of the second medicine is also not particularly limited as long as it is in accordance with a route of administration that develops the action thereof. In general, since the second medicine is administered locally to a tumor, the dosage form, the liquid medium, and the additive agent may be the same as those for the first medicine.

The first medicine and the second medicine may be used at the same time or may be used with a time difference. Independent administration schedules can be set for respective medicines, and respective medicines can be administered to a target in accordance with the schedules. Furthermore, any number of doses can be set for respective medicines, and single or multiple doses can be applied.

A tumor to which the concomitant drug disclosed in the present application is administered is not particularly limited as long as it can be recognized by and infected with an oncolytic virus belonging to the herpes simplex virus type 1 and may be, for example, melanoma, pancreatic cancer, breast cancer, head and neck cancer, ovarian cancer, liver cancer, colorectal cancer, bladder cancer, esophageal cancer, lung cancer, prostate cancer, or the like.

### [Embodiment of Pharmaceutical Composition]

Next, the embodiment of the pharmaceutical composition will be described. In the embodiment of the concomitant drug, the first medicine and the second medicine are used in combination as described above. On the other hand, in the embodiment of the pharmaceutical composition, an oncolytic virus belonging to the herpes simplex virus type 1 and a STING agonist are not considered as separate pharmaceutical compositions, respectively, and a combination of the oncolytic virus and the STING agonist is considered as a single pharmaceutical composition, which makes a difference from the embodiment of the concomitant drug. Further, the pharmaceutical composition of the present invention is administered to a patient in the order of the first medicine and then the second medicine. Other features are the same as those in the embodiment of the concomitant drug.

### [Embodiment of Tumor-Treating Pharmaceutical Composition]

Next, the embodiment of the tumor-treating pharmaceutical composition will be described. As illustrated in Examples and Comparative examples described later, the oncolytic virus belonging to the herpes simplex virus type 1 and the STING agonist can improve respective antitumor effects when used in combination. Thus, the tumor-treating pharmaceutical composition containing the above oncolytic virus as an active component can be used in a use of combined therapy with the tumor-treating pharmaceutical composition containing the STING agonist as an active component, which makes a difference from the embodiment of the concomitant drug, and other features are the same. Further, the tumor-treating pharmaceutical composition containing the STING agonist as an active component can be used in a use of combined therapy with the tumor-treating pharmaceutical composition containing the above oncolytic virus as an active component, which makes a difference from the embodiment of the concomitant drug, and other features are the same.

Although the embodiment disclosed in the present application will be specifically described below with Examples, these examples are provided only for the purpose of illustration of the embodiment and neither limit nor intend to restrict the scope of the invention disclosed by the present application.

### EXAMPLES

### Example 1

### (1) Creation of Tumor-Bearing Mice

Six-week-old female C3H/He Slc mice (purchased from Chubu Kagaku Shizai Co., Ltd.) were used as mice. A tumor of murine squamous cell carcinoma (SCC VII; given by professor Shin-ichiro Masunaga, Kyoto University Institute for Integrated Radiation and Nuclear Science) was cut into 2 mmcubes, and two of the cubes were implanted under the skin on both sides of the abdomen of the mice by a tumor implantation needle (both-side side-abdomen implanted model). When the size of the tumor reached the size of 100 mm³, grouping was performed. Although differing among the mice, one tumor end and the other tumor end were spaced apart from each other by about 10 mm.

### (2) Preparation of Drug

First medicine: C-REV was suspended in a saline solution to obtain a concentration of 1×10⁶ pfu/100 μl.

Second medicine: 2′3′-cGAMP (No. tlrl-nacga23 by Invivogen) was dissolved and suspended in a saline solution to obtain a concentration of 20 μg/100 μl.

### (3) Administration to Tumor

Day 0: The first medicine of 100 μl was administered to a tumor (treated group) on one side of the abdomen. Further, a saline solution of 100 μl was administered to a tumor (non-treated group) on the other side of the abdomen.
Day 3: The second medicine of 100 μl was administered to a tumor (treated group) on one side of the abdomen. Further, a saline solution of 100 μl was administered to a tumor (non-treated group) on the other side of the abdomen.
Day 6: The second medicine of 100 μl was administered to a tumor (treated group) on one side of the abdomen. Further, a saline solution of 100 μl was administered to a tumor (non-treated group) on the other side of the abdomen.

### (4) Measurement of Tumor Size

The tumor size was calculated from multiplication of the longer diameter by the shorter diameter by the shorter diameter by 1/2 of a tumor.

### Comparative Example 1

The experiment was performed in the same procedure as for Example 1 except for administration of a saline solution of 100 μl on Day 3 and Day 6 instead of the administration of the second medicine to the treated group.

### Comparative Example 2

The experiment was performed in the same procedure as for Example 1 except for administration of the second medicine on Day 0 instead of the administration of the first medicine to the treated group and except for administration of a saline solution of 100 μl on Day 3 and Day 6 instead of the administration of the second medicine to the treated group.

### Comparative Example 3

The experiment was performed in the same procedure as for Example 1 except for administration of a saline solution of 100 μl on Day 0 instead of the administration of the first medicine to the treated group and except for administration of a saline solution of 100 μl on Day 3 and Day 6 instead of the administration of the second medicine to the treated group.

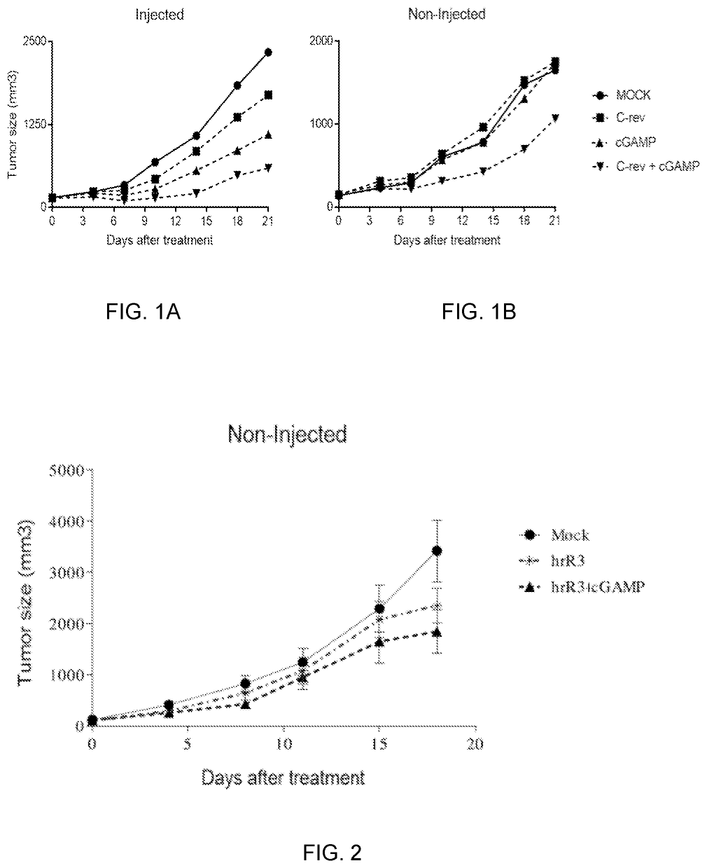

FIG. 1A and FIG. 1B represent graphs illustrating results of measurement of the tumor size in Example 1 and Comparative examples 1 to 3, FIG. 1A is a graph illustrating the result of a treated group with administration of the drug, and FIG. 1B is a graph illustrating the result of a non-treated group without administration of the drug. First, as illustrated in FIG. 1A, it was confirmed that, even when only Comparative example 1 (C-REV) was administered and when only Comparative example 2 (cGAMP) was administered, an antitumor effect was developed compared to Comparative example 3 (MOCK) in which only the administration of the saline solution was administered. As indicated by the result of Example 1 (C-REV+cGAMP), however, it was revealed that the combined use of the C-REV and the cGAMP exhibits a significant antitumor effect.

Further, as illustrated in FIG. 1B, Comparative example (C-REV) and Comparative example 2 (cGAMP) exhibited substantially the same result as Comparative example 3 against the tumor formed spaced apart on the opposite side of the abdomen from the treated group, that is, exhibited almost no antitumor effect against the tumor spaced apart from the treated group subjected to the drug administration. In contrast, in Example 1 (C-REV+cGAMP), the C-REV and the cGAMP were used in combination, and thereby a significant antitumor effect was exhibited against even the tumor spaced apart from the treated group subjected to the drug administration. Note that, although it is said that the C-REV can destroy a tumor cell around an infected tumor cell, the result indicated in FIG. 1B shows that the C-REV exhibited almost no antitumor effect against the tumor formed spaced apart from the tumor subjected to the C-REV administration. In contrast, in Example 1, an antitumor effect was exhibited also against a tumor spaced apart. It is considered that the administration of the first medicine (C-REV) and the second medicine (cGAMP) to a tumor cell caused an unpredictable action mechanism, and the effect that would not be achieved alone was obtained.

From the above results, while the first medicine (C-REV) and the second medicine (cGAMP) each exhibit an antitumor effect alone, the combined use of the first medicine and the second medicine achieves:

(1) an advantageous effect (prominent effect) that an antitumor effect against a tumor subjected to the administration is improved, and
(2) an advantageous effect (different type of effect) that, since an antitumor effect is exhibited against not only a tumor subjected to the administration but also a tumor spaced apart from the tumor subjected to the administration, even when there is a very small tumor that exists around a tumor found by inspection but is unable to be found by the inspection, treatment can also be performed on such an unfound tumor at the same time. Therefore, the combined use of the first medicine and the second medicine can achieve a prominent effect and a different type of effect compared to the advantageous effects achieved by the first medicine and the second medicine, respectively, and compared to a case where respective medicines are used separately.

Further, from the results of Example 1 and Comparative examples 1 to 3 described above, it was confirmed together that (1) a combination of a C-REV and a STING agonist can be used as a pharmaceutical composition, that (2) a tumor-treating pharmaceutical composition containing a C-REV as an active component can be used for a use of combined therapy with a tumor-treating pharmaceutical composition containing a STING agonist as an active component, and that (3) a tumor-treating pharmaceutical composition containing a STING agonist as an active component can be used for a use of combined therapy with a tumor-treating pharmaceutical composition containing a C-REV as an active component.

As described above, it was confirmed that the combined use of the first medicine and the second medicine achieves “a different type of effect that the antitumor effect was exhibited against not only a tumor subjected to administration but also a tumor spaced apart from the tumor subjected to the administration” in addition to the prominent effect that the antitumor effect against a tumor subjected to administration is improved. An experiment was performed in the following procedure to determine as to whether or not the different type of effect is achieved against a tumor spaced apart even when a herpes simplex virus type 1 other than the C-REV is used as the first medicine.

### Example 2

The experiment was performed in the same procedure as for Example 1 except for use of the hrR3 instead of the C-REV as the first medicine.

### Comparative Example 4

The experiment was performed in the same procedure as for Comparative example 1 except for use of the hrR3 instead of the C-REV.

### Comparative Example 5

The experiment was performed in the same procedure as for Comparative example 3.

FIG. 2 is a graph illustrating a result of measurement of the size of a tumor (a tumor to which no drug was administered, corresponding to FIG. 1B) spaced apart from a tumor to which the drug was administered in Example 2 and Comparative examples 4 to 5. As is apparent from FIG. 2, it was confirmed that, in Example 2 in which the hrR3 and the cGAMP were used in combination, a significant antitumor effect was exhibited against a tumor spaced apart from a tumor subjected to the drug administration compared to Comparative example 4 in which the hrR3 was used alone.

### INDUSTRIAL APPLICABILITY

The concomitant drug, the pharmaceutical composition, and the tumor-treating pharmaceutical composition disclosed in the present application exhibit a significant antitumor effect against a tumor subjected to administration and exhibit an antitumor effect also against a tumor spaced apart from the tumor subjected to administration. Therefore, the concomitant drug, the pharmaceutical composition, and the tumor-treating pharmaceutical composition disclosed in the present application are useful for development of pharmaceutical products for tumor treatment in universities, medical institutes, pharmaceutical companies, and the like.

1. A concomitant drug for treating a malignant tumor, the concomitant drug comprising:
a first medicine containing an oncolytic virus belonging to a herpes simplex virus type 1 as an active component; and
a second medicine containing a stimulator of interferon genes agonist as an active component.

2. The concomitant drug according to claim 1, wherein the oncolytic virus is a mutant virus not artificially modified.

3. The concomitant drug according to claim 1, wherein the stimulator of interferon genes agonist is 2′3′-cyclic GMP-AMP.

4. The concomitant drug according to claim 1, wherein the first medicine and the second medicine are administered to a malignant tumor.

5. A pharmaceutical composition for treating a malignant tumor, the pharmaceutical composition comprising, as active components, a combination of:
an oncolytic virus belonging to a herpes simplex virus type 1; and
a stimulator of interferon genes agonist,
wherein the pharmaceutical composition is used to be administered to a patient in order of the oncolytic virus belonging to the herpes simplex virus type 1 and the stimulator of interferon genes agonist.

6. The pharmaceutical composition according to claim 5, wherein the oncolytic virus is a mutant virus not artificially modified.

7. The pharmaceutical composition according to claim 5, wherein the stimulator of interferon genes agonist is 2′3′-cyclic GMP-AMP.

8. The pharmaceutical composition according to claim 5, wherein the oncolytic virus and the stimulator of interferon genes agonist are administered to a tumor.

9. A malignant tumor-treating pharmaceutical composition containing, as an active component, an oncolytic virus belonging to a herpes simplex virus type 1 for use in combined therapy with a malignant tumor-treating pharmaceutical composition containing a stimulator of interferon genes agonist as an active component.

10. A malignant tumor-treating pharmaceutical composition containing, as an active component, a stimulator of interferon genes agonist for use in combined therapy with a malignant tumor-treating pharmaceutical composition containing an oncolytic virus belonging to a herpes simplex virus type 1 as an active component.

11. The concomitant drug according to claim 2, wherein the stimulator of interferon genes agonist is 2′3′-cyclic GMP-AMP.

12. The concomitant drug according to claim 2, wherein the first medicine and the second medicine are administered to a malignant tumor.

13. The concomitant drug according to claim 3, wherein the first medicine and the second medicine are administered to a malignant tumor.

14. The concomitant drug according to claim 11, wherein the first medicine and the second medicine are administered to a malignant tumor.

15. The pharmaceutical composition according to claim 6, wherein the stimulator of interferon genes agonist is 2′3′-cyclic GMP-AMP.

16. The pharmaceutical composition according to claim 6, wherein the oncolytic virus and the stimulator of interferon genes agonist are administered to a tumor.

17. The pharmaceutical composition according to claim 7, wherein the oncolytic virus and the stimulator of interferon genes agonist are administered to a tumor.

18. The pharmaceutical composition according to claim 15, wherein the oncolytic virus and the stimulator of interferon genes agonist are administered to a tumor.
